(19) United States
(12) Patent Application Publication
(10) Pub. No.: US 2008/0077193 A1
(43) Pub. Date: Mar. 27, 2008

(54) SCAVENGED ENERGY FOR ELECTRIC FIELD GENERATION TO PREVENT BONE LOSS AND ENCOURAGE BONE GROWTH FOR ORTHOPEDIC APPLICATIONS

(75) Inventors: Jennifer K. Bow, Philadelphia, PA (US); Joshua M. Pearce, Philadelphia, PA (US)

Correspondence Address: Jennifer Bow, 834 Chestnut St. Apt 1428, Philadelphia, PA, 19107, US

(73) Assignee: Jennifer Bow, Philadelphia, PA (US)

(21) Appl. No.: 11/861,256

(22) Filed: Sep. 25, 2007

Related U.S. Application Data
(60) Provisional application No. 60/847,054, filed on Sep. 26, 2006.

(51) Int. Cl. A61N 1/00

(52) U.S. Cl. 607/51

(57) ABSTRACT

In this invention, stress shielding effects seen in total joint arthroplasty patients and trauma patients is prevented by causing the bone above the press fit of the implant to continue to act as if it were under stress by inducing an electric field in the surrounding bone that simulates the electric field generated when bone is under mechanical stress. This same effect is used to stimulate bone union in fractures and surgical arthrodesis, as well as to encourage increased bone mass and consolidation of bone during limb lengthening procedures. The electric field can be generated by a number of methods utilizing scavenged energy, with the preferred embodiment being the generation of electricity using arrays of thousands of thermoelectric generators built into an implantable chip. These generators exploit the well-known thermocouple effect, in which a small voltage is generated when two of the junctions between two dissimilar materials are kept at different temperatures. The temperature gradient is produced between the underside of the skin and the interior of the body.

### CROSS REFERENCE TO RELATED APPLICATIONS

This application claims the benefit of provisional patent application Ser. No. 60/847,054 filed Sep. 26, 2006 by the present inventors.

### FEDERALLY SPONSORED RESEARCH

Not applicable

### SEQUENCE LISTING OF PROGRAMS

Not applicable

### BACKGROUND OF THE INVENTION

1. Field of the Invention

This invention relates to devices used to prevent bone damage from stress shielding in orthopedic implants, as well as stimulation of bone healing and bone growth, specifically to such devices which use scavenged energy to create an artificial electric field mimicking those existing naturally in normal bones.

2. Prior Art

In orthopaedic surgery, bone is often manipulated and fixed with metal plates or during total joint replacement surgeries, implants are inserted into the bone that have a different modulus of elasticity as the surrounding bone. This difference in the elasticity of the plates or implants from the surrounding bone creates a situation in which the bone is being offloaded in terms of the mechanical stress that the bone is exposed to, since the metal is then bearing the larger portion of mechanical stress acting through that segment. The bone that is offloaded from the mechanical stress thus becomes weaker over time, and develops less bone mass. This phenomenon is referred to as stress shielding. A method is needed to provide bone support without causing stress shielding of the bone proximal to the press-fit of the prosthesis in orthopedic implants and to the bone that is adjacent to orthopaedic plates. Solutions to combat stress shielding have focused on using materials that have a modulus of elasticity closer to that of cortical bone (titanium has a modulus of elasticity closer to cortical bone than does stainless steel or cobalt-chrome), and changing implant design to more tapered stems in order to produce less of a distal press-fit, and hence less stress shielding. Unfortunately, these strategies do not successfully tackle every situation of stress shielding since revision implants often require distal press-fit in order to provide adequate stability and isthmic fit for the prosthesis. This leaves important areas of bone (the greater trochanter in the case of total hip arthroplasty [THA]—where the abductor musculature attaches) offloaded in terms of mechanical stress and as such, this bone weakens and can ultimately disappear. A method is needed to provide bone support without causing stress shielding of the bone proximal to the press-fit of the prosthesis.

Feedback for bone growth is explained by Wolff's Law: bone grows in response to mechanical stress. One solution to bone degeneration from stress shielding, therefore is to increase the mechanical stress on the bone. Another solution must be derived to induce the same effects as the mechanical stress. One theory for the mechanism behind Wolff's Law is that load-induced piezoelectric potentials in bone, induced by deformation of collagen, provide a means by which stress or strain intrinsically alter the biophysical environment of the bone cell. There are also relatively large electrokinetic currents (streaming potentials) produced by the strain-induced flow of charged constituents of extracellular fluids flowing past the mineral phase of the material. These electric currents underlying the mechanical stress observed are a secondary target for feedback regulation. Measurement of the electric potentials generated by the functional levels of strain shows the average field intensities in bone are on the order of 1 μV/μe (micro volt per micro strain). Adult skeletons are seldom subject to strains exceeding 4000 μe. So if “endogenous” fields are to influence bone morphology they must do as at field intensities below 4 mV/cm. The larger percentage of bone tissue is rarely subject to strains greater than 500 μe, yet bone mass is retained in these areas. So we need to produce fields around 500 μV/cm (Buckwalter, J., Einhom, T., and Simon, S., (editors) Orthopaedic Basic Science: Biology and Biomechanics of the Musculoskeletal System, 2^{nd} edition, American Academy of Orthopaedic Surgeons, 2000).

It has already been determined that exogenously-applied microamperes, direct electrical currents, capacitatively-coupled electric fields, and alternating, pulsed electromagnetic fields (PEMF) affect bone cell activity in various ways in culture, in living bone tissue and clinically, in fracture nonunion (Buckwalter, J., Einhorn, T., and Simon, S., (editors) Orthopaedic Basic Science: Biology and Biomechanics of the Musculoskeletal System, 2^{nd} edition, American Academy of Orthopaedic Surgeons, 2000). PEMF is approved by the FDA for clinical use in fracture nonunions as a method to noninvasively induce electric currents to replace the endogenous bone currents in the absence of the normal mechanical loading. Normally a magnetic field is induced by forcing electric current through a wound wire coil placed over the fracture. Electrical currents proportional to the time rate of change of the magnetic flux traverse the fracture as a result of Faraday induction. A second method is to use time-varying electric currents in a fracture using capacitive coupling, in which time-varying electric field is applied to the limb by means of capacitor plates placed on the skin overlying the fracture. For example—McLeod and Rubin used sinusoidally varying fields to stimulate bone remodeling activity. They found that extremely low frequency (ELF) sinusoidal electric fields (below 150 Hz) are quite effective at preventing bone loss and inducing new bone formation. Field effectiveness peaks in 15-30 Hz range and there is a strong frequency selectivity. At 15 Hz field induced electric fields of no more than 1 μV/cm affected remolding activity (McLeod K J. Rubin C T: The effect of low frequency electrical fields on osteogenesis. J. Bone Joint Surg. 1992; 74A:920-929). Thus in the current invention a device that can create electric fields at 4 mV/cm at frequencies below 10 Hz is necessary.

As these traditional sources of electric field are exogenously applied, meaning that the patients must purchase expensive equipment (or travel to a medical office that has such a device) and spend long amounts of time at or near a machine to provide the power to produce the necessary electric fields, this is extremely inconvenient and is likely to result in non-compliance with an optimized regime of electric field exposure. An improved system/device for protecting bones from stress shielding or encouraging the healing and strengthening of bones by producing the necessary electric fields would be implanted within the body.

There are several prior inventions that anticipate this need and attach electrodes in some way within the body, unfortunately they are hampered by the use of an external power supply. These inventions use an external source of energy to provide the power to create the current to stimulate tissue growth. The early models are somewhat crude, such as U.S. Pat. No. 4,549,546 Bone growth stimulator is external to the prosthetic and involves an external source of power. U.S. Pat. No. 6,034,295 Implantable device having an internal electrode for stimulating growth of tissue and U.S. Pat. No. 4,549,547 Implantable bone growth stimulator, are other examples of this thinking. Other patents give detailed information for monitoring the implanted tissue stimulator such as U.S. Pat. No. 5,766,231 Implantable growth tissue stimulator and method of operation, or U.S. Pat. No. 5,565,005 Implantable growth tissue stimulator and method operation. Others also have complicated electronics for implanting outside of the spine such as U.S. Pat. No. 5,441,527 Implantable bone growth stimulator and method of operation. All of these patents still suffer from the need to use an external source of power to create the electric field that is generated by the device within the body. This again is inconvenient as the patient would need to carry this bulky source of power around with them or dedicate long periods of every day to visiting a fixed source of power. An improved device would provide the needed electric fields with no external sources of power at all.

U.S. Pat. No. 5,738,521 Method for accelerating osseointegration of metal bone implants using electrical stimulation, identifies this need. Although it is used primarily for dentistry implants a power source along with the electronics and wires is mentioned in the disclosure. Unfortunately, this power source is a battery to run current to the implant and surrounding tissue. The battery is not in the implant and also will suffer from the problem of extremely limited lifetimes. When the battery runs out of power the patient must undergo another expensive and potentially dangerous surgery to have the battery changed out. In orthopedic applications this is particularly problematic. An improved device/system would provide its own electric field from a source of energy that did not run out such as a battery.

This was anticipated by U.S. Pat. No. 6,143,035 Implanted bone stimulator and prosthesis system and method of enhancing bone growth. This patent uses implantable piezoelectrics to power devices like pacemakers or to help with bone growth. This patent is primarily concentrating on healing fractures and is limited to only using piezoelectrics as an energy source. The use of piezoelectrics for the energy source is a major drawback of this patent. First, the electric field will not be continuously applied, or even applied for a large fraction of time in a day—because it will only provide an electric current when the piezoelectric devices are under stress. This is particularly unhelpful for those suffering lower limb fractures or those undergoing total joint replacement as they already have limited mobility (and thus are not moving in ways to stress the piezoelectric device and provide stimulation for their bones). Similarly, when the patient that is mobile is resting or sleeping and not putting any stress on the piezoelectric device, there will be no current and thus no electric field to stimulate the bone. Thus this device is the least helpful to the largest fraction of possible users. It will thus not provide the optimal bone stimulation and defense against stress shielding in orthopedic implants. It also requires a second surgical site for the implantation of the piezoelectric device. Finally, and most importantly, for such a device to work (it must deform under mechanical stress and recoil), it must also be offloading the bone where it is placed to a certain extent. The bone that it is anchored to it, will thus be stress shielded. Thus it could potentially be creating more of the same problem it was meant to solve in a different location.

In summary, all the devices used to prevent bone damage from stress shielding in orthopedic implants, as well as stimulation of bone healing and bone growth using electric fields heretofore known suffer from a one or more of the following disadvantages:
(a) The means to generate the electric field was limited to devices external to the body and thus were:
   a. cumbersome,
   b. inconvenient,
   c. limited the patient's movement
   d. expensive,
   e. used large amounts of electric power because the electric field strength is inversely proportional to the square of the distance from the source.
   f. or a combination of the above.
(b) The means to power the internal device that creates the electric field were limited to devices external to the body and thus were:
   a. cumbersome,
   b. inconvenient,
   c. limited the movement of the patient
   d. expensive,
   e. used large amounts of electric power because the electric field strength is inversely proportional to the square of the distance from the source.
   f. or a combination of the above.
(c) A device powered by a limited life battery. When the battery runs out of power the patient must undergo another expensive and potentially dangerous surgery to have the battery removed or replaced.
(d) The electric field will not be continuously applied, or applied very infrequently, and thus will not be stimulating the bone optimally.
(e) A device powered by piezoelectrics. This is has several drawbacks including:
   a. It will not provide an electric field continuously and will be useless when the patient is resting or sleeping.
   b. This effect will be aggravated by patients with limited mobility and the device is useless for patients with no mobility.
   c. It requires a second surgical site or longer incision and dissection for the implantation of the piezoelectric device, which increases the chance of infection and the patient's postoperative discomfort, as well as potentially increasing the devascularization of the bone in question.
   d. This device would not be useful without gravity or weight bearing on the bone, and thus would not be useful in space and would have limited efficiency underwater.
   e. For such a device to work (it must deform under mechanical stress and recoil), it must also be offloading the bone where it is placed to a certain extent, thus the problem it is meant to prevent—stress shielding—can actually be caused by the device.

An ideal implant would contain a means of creating its own electric field to stimulate the bone and prevent stress shielding and this electric field would be applied as often as possible. The current invention overcomes all of the limitations of the prior art by utilizing scavenged energy within the human body to provide power (often continuous) to a device to create an electric field to stimulate the bone and protect against bone loss surrounding orthopedic implants.

### OBJECTS AND ADVANTAGES

Several objects and advantages of this invention are:
(a) to provide a means to generate the electric field from devices inside the body and thus are:
   a. inconspicuous,
   b. convenient,
   c. near the target site and thus use little energy,
   d. in no way limit the mobility of the patient,
   e. made inexpensively and from relatively small amounts of materials;
(b) to provide a means to power the internal device that creates the electric field that is also internal to the body so it is:
   a. inconspicuous,
   b. convenient,
   c. near the target site and thus use little energy,
   d. in no way limit the mobility of the patient,
   e. made inexpensively and from relatively small amounts of materials;
(c) to provide a means for the device to be fabricated in a way that the device lifetime will be greater than the patient lifetime so that it never needs to be removed or replaced;
(d) to provide a power source that will not “run out”;
(e) to provide a continuous electric field, and thus will be stimulating the bone continuously;
(f) to provide a device that operates when the patient is sleeping, unconscious, or resting;
(g) to provide a device that operates when the patient has no mobility;
(h) to provide a device that can be implanted in the same site as the orthopedic implant;
(i) to provide a means for generating the electric current that can be contained within the implant itself,
j) to provide a system that is functional in space;
(k) to provide a system that is functional underwater;
(l) to provide a system that requires no maintenance;
(m) to provide a means of generating the electric field without offloading any bones in the body of the patient.

In this invention and its embodiments, bone is electrically stimulated, similarly to its normal electric stimulation when a person is walking on it, thus improving fracture healing, bone consolidation and preventing stress shielding caused by total joint arthroplasty by encouraging the bone above the press fit of the implant to continue to act as if it were under stress by inducing an electric field in the surrounding bone that simulates the electric field generated when bone is under mechanical stress. The electric field can be generated by a number of methods discussed below with the preferred embodiment being the generation of electricity using arrays of thousands of thermoelectric generators built into an implantable chip. These generators exploit the well-known thermocouple effect, in which a small voltage is generated when two of the junctions between two dissimilar materials are kept at different temperatures. The temperature gradient is produced between the underside of the skin and the interior of the body.

Here we will describe the invention embodied in a THA but this invention comprises applications to other total joint implants, implantation to encourage fracture union in trauma, implantation to encourage fusions after surgical arthrodeses, and implantation to encourage bone consolidation during limb lengthening procedures.

### SUMMARY OF INVENTION

In accordance with the present invention, a device to stimulate bone union in fractures, surgical arthrodesis, bone consolidation and to prevent stress shielding by causing the bone above the press fit of the orthopedic implant to continue to act as if it were under stress, comprises an implantable device for generating electrical current from scavenged energy sources, wire to carry that current to the area of bone to be stimulated and a coil or electrodes to produce an electric field over that area.

### DRAWINGS

### Brief Description

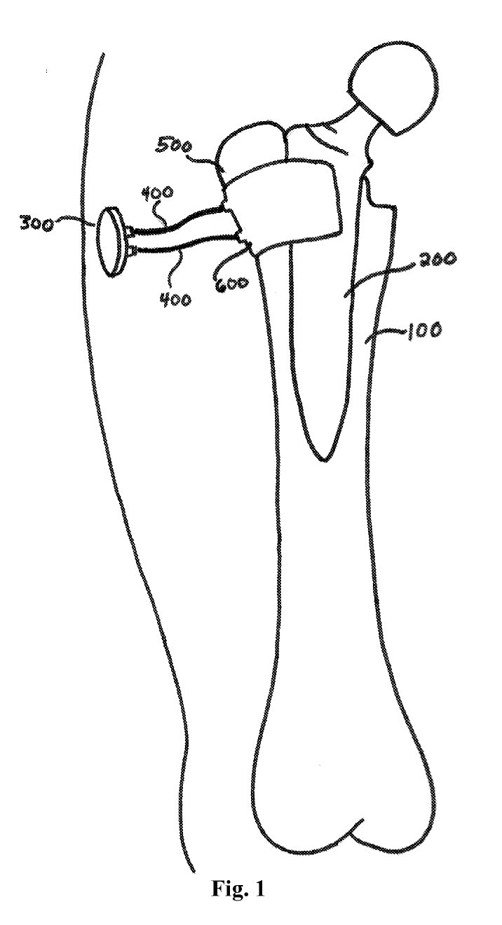

FIG. 1A device utilizing an array of thermocouples to provide current for electrodes placed on bone proximal to the press-fit of the prosthesis to prevent stress shielding.

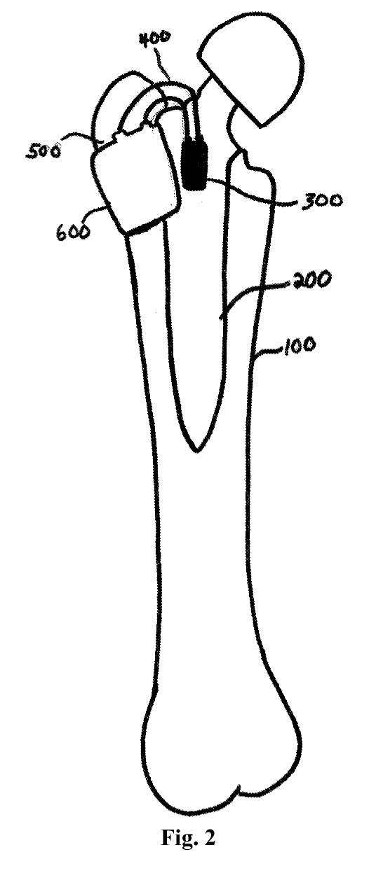

FIG. 2 An orthopedic implant containing a source of scavenged energy to provide current for electrodes placed on bone proximal to the press-fit of the prosthesis to prevent stress shielding.

### DETAILED DESCRIPTION

### FIG. 1

### Preferred Embodiment

The femoral total hip arthroplasty component (200) is implanted into the femur bone (100) which then grows into the porous coating of the implant, thus providing stability for the implant, but creating an area of bone subject to stress shielding (500) since it is no longer subject to the regular mechanical stresses of load-bearing, and thus becomes less dense. The implantable chip containing a thermocouple array (300) is implanted beneath the skin surface at the area of the surgical incision with wires (400) directly connecting this chip producing an electric current with the coil of wire or other electrode (600) placed over the site of stress shielding (500).

### OPERATION

### FIG. 1

The implantable chip (300) in the preferred embodiment will generate electricity using arrays of thousands of thermoelectric generators built into the implantable chip. These generators are thermocouples, in which a small voltage is generated when two of the junctions between two dissimilar materials are kept at different temperatures. The technology is based on application of the Seebeck effect (also known as the Peltier-Seebeck Effect), in which the temperature gradient between the two sides of a Peltier junction generates an electrical current across the junction. There are several commercial thermocouple devices on the market that have the necessary power conversion efficiencies to produce the electric fields necessary for this invention. The temperature gradient between the inner body and the area just under the skin where the chip will be implanted provide the energy available for scavenging. This energy is converted into electrical energy and the electrical current travels down insulated wires (400) to electrodes (600). The electrodes (600) can be as simple as a serpentine wire encapsulated in a flexible polymer resistant to degradation within the body. The electrodes are placed over the area of bone (500) above the press fit of bone proximal to the press-fit of the prosthesis (200) to prevent stress shielding. In FIG. 1 showing the case of total hip arthroplasty, (600) is the greater trochanter where the abductor musculature attaches to the femur bone (100). As electric current passes through the wire an electric field is generated, which matches the natural electric field created when bone is placed under stress. This electric field then stimulates the bone to continue to grow and remodel as if it were subject to normal mechanical stresses.

This device can be implanted in the body during the orthopedic surgery or can be implanted afterwards if stress shielding becomes a concern. Similarly this device can be implanted to protect bone from other kinds of orthopedic implants, stimulate bone healing and assist bone growth in any of the bones in the human or animal body.

### FIG. 2

The femoral total hip arthroplasty component (200) containing a source of scavenged energy (300) is implanted into the femur bone (100) which then grows into the porous coating of the implant, thus providing stability for the implant, but creating an area of bone subject to stress shielding (500) since it is no longer subject to the regular mechanical stresses of load-bearing, and thus becomes less dense. Insulated wires (400) directly connect, through holes in the implant (200), the scavenged energy source (300) producing an electric current with the coil of wire or other electrode (600) placed over the site of stress shielding (500).

### FIG. 2

In this embodiment one of several devices (300) for scavenging energy and converting it into electrical energy (discussed below) is located within an orthopedic implant (200) the electric fields necessary for this invention. This electrical current travels down insulated wires (400) to electrodes (600). The electrodes (600) can be as simple as a serpentine wire encapsulated in a flexible polymer resistant to degradation within the body. The electrodes are placed over the area of bone (500) above the press fit of bone proximal to the press-fit of the prosthesis (200) to prevent stress shielding. In FIG. 2 showing the case of total hip arthroplasty, (600) is the greater trochanter where the abductor musculature attaches to the femur bone (100). As electric current passes through the wire an electric field is generated, which matches the natural electric field created when bone is placed under stress. This electric field then stimulates the bone to continue to grow and remodel as if it were subject to normal mechanical stresses.

This device must be implanted in the body during the orthopedic surgery itself because it is directly integrated within the orthopedic implant. Many other orthopedic implants besides those for THAs are potentially improved by this invention. This embodiment can also be used for improved orthopedic nails to stimulate bone healing and assist bone growth in any of the bones in the human or animal body. The advantage of this embodiment is that the source of energy to drive the electric field can be completely contained within the implant itself and even the electrodes/coils (600) can be fabricated to be part of the implant (200). This invention could then undergo the exact same orthopedic surgery as normally undergone for the application and thus no extra placement of wires or electrodes would be necessary.

### ADDITIONAL EMBODIMENTS

The preferred embodiment of the invention describes the application in a THA, but this invention comprises applications to other total joint implants, implantation to encourage fracture union in trauma, implantation to encourage fusions after surgical arthrodeses, and implantation to encourage bone consolidation during limb lengthening procedures. In all of these additional applications the preferred embodiment would only be altered in minor ways (e.g. lengthening electrical leads or placement of components in the body). The preferred embodiment used for these other applications has the same advantages.

The preferred embodiment of the invention described a thermocouple based power source to drive the electric field. This invention comprises a system to create an electric field using but not limited to the other following scavenged energy sources.

The Dorn effect is the formation of a potential difference between electrodes spatially separated and submerged in a colloidal solution (suspension). In this embodiment, as could be used in FIG. 2, a solution would be contained in a hollowed out area within the implant. A device producing electricity with the Dorn effect could be component (300) in FIG. 2. Due to gravity on earth, vibration (e.g. traveling on a bumpy road), centrifugal force (as in a space station), and the like, the particles suspended in the liquid move from one electrode to the other. The double layer on the boundary between the liquid and each solid particle is smeared out due to the movement of the particles. As a result, the particles become electrically charged. When the charged solid particles settle, a sedimentation potential arises between the electrodes. The potential is can be used to drive an electrical current and create an electric field around the bone in target areas (500). The conditions for the success of this embodiment are that the particles should be suspended in a liquid and the colloidal particle and its surrounding liquid should form a double electric layer. The patient could recharge the Dorn cell by sleeping with an elevated leg (in the case of a THA or knee replacement). For other applications a similar change in orientation would be necessary.

A similar embodiment could take advantage of inertial energy scavenging. In this way a weight that swings as you move can drive a tiny generator. This inertial energy system can be placed in the implant itself as in FIG. 2 component (300). Also, while gravity is absent in space, inertia is not, so this would work for astronauts as well. This type of technology has already been popularized in certain models of Seiko watches.

There are several other methods too numerous to list here, to produce electricity with movement of the body, or kinetic to electrical energy conversion, (e.g. a magnetic moving past a coil of wire) that can also be utilized in the same way as component (300) within this invention. It is important to note, that these methods do not need to generate a lot of electricity in order to be useful in this invention.

A thermophotovoltaic (TPV) system could also be used to create the necessary electric field. A basic TPV system consists of a thermal emitter and a photovoltaic diode cell. The temperature of the thermal emitter normally is above the range at which any form of human application would be possible, but in principle TPV devices can extract energy from any emitter with temperature elevated above that of the photovoltaic device (forming an optical heat engine). For conventional TPV temperatures, this radiation is mostly at near infrared and infrared frequencies. The photovoltaic diodes can absorb some of these radiated photons and convert them into free charge carriers, thus electricity to drive the electric fields to stimulate the bone. Current research in TPV aims at increasing the system efficiencies, which could produce a viable energy source for the system described here.

This invention can also be powered by other scavenged energy sources not listed here as examples, but known to those skilled in the art, which can be built into orthopedic implants as in FIG. 2. or surgically implanted within the body such as in FIG. 1.

### CONCLUSIONS RAMIFICATIONS AND SCOPE

Accordingly, the reader will see that a device used for inducing an electric field in bone from scavenged energy sources of this invention can be used to electrically stimulate any bone in order to improve fracture healing, encourage surgical arthrodesis or bone consolidation and preventing stress shielding caused by total joint arthroplasty. This invention will help patients heal faster, live longer, prevent painful and dangerous revision surgeries, and improve the practical lifetime of orthopedic implants by preventing bone around them from deteriorating.

Furthermore, the (invention) has the additional advantages that
it permits the healing of bone in an inconspicuous and convenient fashion using an internal device that is powered internally,
it permits the healing of bone with no large sources of energy and the concomitant greenhouse gas emissions from the standard energy sources,
it permits the patient to maintain full mobility and partake in any activity to which the patient would normally be able to do, including ‘no activity’ or resting,
it permits the implant to stay in place longer and for the device of the invention to never be removed or replaced,
it permits a continuous electric field, and thus will be both protect the bone continuously and heal the bone faster from intermittent energy supplies for electric fields,
it permits the implantation of the device in the same site as the orthopedic implant and can also be completely integrated into the conventional implant,
it permits the patient to still utilize the device in space, underwater or low gravity environments,
it permits the healing of bone and the protection of stress shielding with no maintenance of the device,
it permits the generation of an electric field without, in any way, contributing to stress shielding of any bone in the body.

Although the description above contains many specifications, these should not be construed as limiting the scope of the invention but as merely providing illustrations of some of the presently preferred embodiments of this invention. For example, the electrodes can have other shapes than what is mentioned; the scavenged energy can be converted to electric fields in other manners than have been described; the invention can be used in non-humans, and the invention can be used in multiple applications (such as total knee arthroplasty, total elbow arthroplasty, bone adjacent to plates used to fix fractures, and other implants not listed here) involving any bone in the body. The method can also be used to encourage bone growth or healing in the setting of fractures and fracture nonunions or delayed unions, and to encourage bony fusion after surgical arthrodesis. There may also be applications to bone consolidation in surgical limb-lengthening procedures.

The invention being thus described and illustrative embodiments illustrated herein, further variations and modifications will occur to those skilled in the art, and all such variations and modifications are considered to be within the scope of the invention as defined by the claim appended hereto and equivalents thereof.

We claim:

1. An internal medical device for stimulating bone comprising:
(a) a means of converting scavenged energy to electricity,
(b) a plurality of electrically conductive wires,
(c) at least one electrode with the ability to create an electric field,
(d) said means of converting scavenged energy to electricity connected by said wires to said electrode to create an electric field in the bone.

2. The device in claim 1 wherein the means of converting scavenged energy to electricity makes use of a thermocouple.

3. The device in claim 1 wherein the means of converting scavenged energy to electricity uses the Dorn Effect.

4. The device in claim 1 wherein the means of converting scavenged energy to electricity uses an inertial energy converter.

5. The device in claim 1 wherein the means of converting kinetic energy of motion to electrical energy.

6. The device in claim 1 wherein the means of producing electricity makes use of a thermophotovoltaic device.

7. The device in claim 1 wherein the means of producing electricity uses a plurality of energy conversion methods.

8. The device in claim 1 wherein the bone stimulation is used to improve bone mass.

9. The device in claim 1 wherein the bone stimulation is used to prevent stress shielding in orthopedic implants.

10. The device in claim 1 wherein the bone stimulation is used to induce bone healing.

11. The device in claim 1 wherein the bone stimulation is used to induce bone consolidation.

12. The device in claim 1 wherein said wire acts as the electrode by locating near the targeted bone.

13. An orthopedic implant containing a means of stimulating bone comprising:
(a) a means of converting scavenged energy to electricity,
(b) at least one electrically conductive wire,
(c) at least one electrode with the ability to create an electric field,
(d) said means of converting scavenged energy to electricity connected by said wires to said electrode to create an electric field at the site of stress shielding.

14. The device in claim 13 wherein the means of converting scavenged energy to electricity uses the Dorn Effect.

15. The device in claim 13 wherein the means of converting scavenged energy to electricity uses an inertial energy converter.

16. The device in claim 13 wherein the means of converting kinetic energy of motion to electrical energy.

17. The device in claim 13 wherein the bone stimulation is used to improve bone mass.

18. The device in claim 13 wherein the bone stimulation is used to prevent stress shielding in orthopedic implants.

19. The device in claim 13 wherein the bone stimulation is used to induce bone healing.

20. The device in claim 13 wherein the bone stimulation is used to induce bone consolidation.
